(19) United States
(12) Patent Application Publication
(10) Pub. No.: US 2023/0022069 A1
(43) Pub. Date: Jan. 26, 2023

(54) PASSIVE SAMPLER DEPLOYMENT HOUSING

(71) Applicant: LUXEMBOURG INSTITUTE OF SCIENCE AND TECHNOLOGY (LIST), Esch-sur-Alzette (LU)

(72) Inventors: Denis PITTOIS, ROEDGEN (LU); Tom GALLÉ, ESCH-SUR-ALZETTE (LU)

(21) Appl. No.: 17/788,047

(22) PCT Filed: Dec. 17, 2020
(86) PCT No.: PCT/EP2020/086649

§ 371 Date: Jun. 22, 2022

(30) Foreign Application Priority Data
Dec. 23, 2019 (LU) LU101569

(51) Int. Cl. G01N 1/20

(52) U.S. Cl. CPC G01N 1/20

(57) ABSTRACT

A passive sampler deployment housing, the housing comprising a channel for guiding a stream flow of a liquid in a main direction, the channel having two side walls and one downstream wall, defining a sampling chamber; at least one support means adapted for supporting at least one detecting means and arranged in the channel, the downstream wall, disposed essentially perpendicular to the liquid main direction, having a height such that the flow of the liquid is accumulated in the channel at a level that is sufficient to overlap the support means and optionally the detecting means. The invention also relates to a device including the housing of and at least one detecting means, the at least one detecting means comprising a frame including two membrane supporting means, and a membrane, comprising a sorbing material, which is clamped therebetween.

The present invention is the US national stage under 35 U.S.C. §371 of International Application No. PCT/EP2020/086649 which was filed on Dec. 17, 2020, and which claims the priority of application LU101569 filed on Dec. 23, 2019 the contents of which (text, drawings and claims) are incorporated here by reference in its entirety.

### FIELD

The present invention relates to liquid sampling and more particularly to passive sampler deployment housing for contaminant monitoring.

### BACKGROUND

Until recently, only the southern countries were facing problems of water scarcity, but nowadays these problems started to spread to the northern regions. Indeed, the precarious balance between water demand and its availability has reached a critical point in many countries because of reserve overexploitation and periods of low rainfall and even drought. The decrease of river flows and groundwater levels also implies a diminution of the water quality due to a lesser dilution of the pollutants (EEA, 1999, Sustainable water use in Europe - Part 1: Sectoral use of water).

The chemical pollution of the water can have several origins depending on the anthropogenic activities occurring in its vicinity. By the usage of pesticides, e.g., herbicides, insecticides, fungicide etc., the agricultural activities can lead to the release of these compounds to the groundwater depending on their mobility. What is more, due to different mechanisms, the parent compounds can go under some transformation/degradation caused by microorganisms present in the soil or chemical reactions like hydrolysis or oxidation and if they cannot be detected anymore, their metabolites are.

The rivers can also be contaminated by farming via runoff water from the fields carrying the pesticides and their metabolites as well. The urban activities have an impact on the water quality either indirectly by impervious surface runoffs carrying compounds such as heavy metals, oils, gasoline, herbicides for weed control in the streets, or directly from wastewater treatment plants effluents or from the combine sewer overflows. The actual design of most of the wastewater treatment plants do not allow a full degradation of xenobiotics and the concentration of micro-pollutants like pharmaceuticals, oestrogens and biocides, in the effluent can be as high as in the influent. Wastewater treatment plants are actually considered as an important path for pollutants to river waters. Historic landfills, even if refurbished, can be a source of diffuse pollution with the leaching of heavy metals or polyaromatic hydrocarbons to river or groundwater.

Individually, the presence of some of these chemical compounds in water can potentially have a disastrous effect to the aquatic ecosystems and human health even if, for some like the oestrogens, their concentrations are in the nanogram per litre range. However, the combination of all of these chemicals may lead to synergetic interactions increasing their combined toxicity, which is far more problematic (Sousa, J.C.G., et al., A review on environmental monitoring of water organic pollutants identified by EU guidelines. Journal of Hazardous Materials, 2018. 344: p. 146-162).

To establish spatial distributions and time evolutions of the chemical water pollution, monitoring programs can rely on different sampling techniques. On one hand, there is the active technique consisting of local sample grabbing at different frequencies from monthly to hourly depending on the studied system. This technique may also rely on the installation of programmable autosamplers to monitor shorter events like flood waves. This technique may require numerous of samples over the campaign period to establish sound time series on specific sampling sites. Ultimately, this technique can be expensive.

On the other hand, there is the passive sampling technique consisting of the accumulation of pollutants into a collecting medium during a defined exposure time (see US 6 478 961B2, CN107462435A and WO2017177099A1). The advantages of this method are firstly that the concentration of the pollutants will be integrated over the exposure time giving a more realistic view on their presence and concentration level than several discrete samples catching or missing any concentration variation. Secondly, lower detection limits can be reached and finally the compounds are less prone to degrade once adsorbed on the collecting medium or absorbed in it (Namiesnik, J., et al., Passive sampling and/or extraction techniques in environmental analysis: a review. Analytical and Bioanalytical Chemistry, 2005. 381 (2): p. 279-301). The drawbacks of the passive sampling are firstly that you need to determine the sampling rate of each pollutant in order to back calculate their time weighted average concentrations from their accumulated masses and, secondly, that their accumulation rates are low and therefore the exposure time should be planned in consequence. The compound measurement success is dependent on the combination of several factors: its concentration, its limit of quantification, its sampling rate and the peak duration of the event.

Some of the passive samplers are based on the sequestration of a sorbing agent (e.g. modified polymeric resin, C18 disk etc.) by two microporous membranes (e.g. polyethersulfone, glass microfibers etc.) secured by two stainless steel washers. The driving force of the compounds accumulation in the passive sampler is the presence of a gradient of diffusion between the sampling environment and the sorbing agent.

Since the membranes are quite fragile, this kind of passive samplers should be mounted on a housing to ensure their protection by avoiding their perforation and thus the loss or the damage of the sorbing material. The commercially available housings have a cylindrical shape with mesh-type permeability and can host several passive samplers at the same time. The mesh size of these housings protects the passive samplers from clogging by large to medium size foreign materials. Some embodiments can also directly combine the housing and the sampler into one single entity (see CN201520157153 and RU2384833C1).

If they are well suited for large or deep sampling environments, the size of this kind of housing can be a disadvantage when used in some locations like a low flow system and more generally in shallow water where the full immersion of the passive samplers may be difficult or even impossible. For that reason, some custom-made housings have been developed in order to fit in sampling environments as wells or pipes (see US20140290391 A1, US005942440A, DE102016003843B3 and CN205858336U) and some others for shallow sampling environments (USD734127S and CN107636441A).

Unfortunately, those designs do not guarantee that the passive samplers stay always immersed in the sampling environment during the whole exposure time. Even if they are immersed during their installation, there is always a risk that they experience dry periods where both membranes and sorbing agent may lose their hydration. In this case, when the passive samplers are again immersed, the hydration of the membranes may create a flush of water and pollutants towards them and the sorbing agent, increasing therefore the initial compound uptake rate (Bailly, E., et al., Calibration and field evaluation of polar organic chemical integrative sampler (POCIS) for monitoring pharmaceuticals in hospital wastewater. Environmental Pollution, 2013. 174: p. 100-105). If repeated several times during the exposure period, it will lead to a wrong interpretation of their accumulated masses.

Another disadvantage is the mesh size of these commercially available housings. If it is able to block large to medium size foreign materials, when working in a sampling environment with a high load of suspended matter, the membranes of the passive samplers may be clogged resulting in a decrease of the pollutant uptake rate.

### SUMMARY

In order to overcome at least one of cited drawbacks, the present invention provides a passive sampler deployment housing, the housing comprising:
1) a channel for guiding a stream flow of a liquid in a main direction, the channel having two side walls and one downstream wall, defining a sampling chamber;
2) at least one support means adapted for supporting at least one detecting means and arranged in the channel, the downstream wall, disposed essentially perpendicular to the liquid main direction, having a height such that the flow of the liquid is accumulated in the channel at a level that is sufficient to overlap the at least one support means and optionally the at least one detecting means.

According to various embodiments of the invention, it is possible to efficiently manage the housing for determination and quantification of pollutants in a stream of a liquid flow, like rivers and other water streams, because the housing is able to include at least one detecting means, the latter being immersed in the stream flow of the liquid owing to the downstream wall, which is arranged to retain the stream flow of the liquid, allowing the efficient recovery of the pollutants.

The housing can also include an inlet, disposed upstream of the channel, the housing comprising a mesh screen delimiting an upper end and a lower end that can have a V-shaped form presenting two angular walls, the angle between the two walls can be of from 45° to 80°, in various instances of from 60° to 80°. An angle of 60°-80° provides the better flow of the liquid throughout the channel.

Alternatively, the mesh screen can have a ∩ shape or an arc of circle shape.

The inlet can also include an upstream wall disposed at an upstream position with regard to the at least one support means. In various instances, the upstream wall is perpendicular to both side walls of the channel.

The housing with the inlet and the mesh screen provides the retention of undesirable materials that can be present in the liquid flow, such as sediments, polymeric waste, parings and scrap, vegetables and/or trees leaves and roots and the like. The at least one detecting means can then not be clogged enough to impair sorbing properties thereof.

In various instances, the mesh screen is removable from the housing. This allows easy cleaning of the mesh screen.

In various instances, the mesh screen can include at least one positioning means.

Advantageously, the mesh screen with the upstream wall are defining a sedimentation trap, the upper end of the mesh screen having a height that is greater than the height of the upstream wall. Such a disposition enhances the retention of water in the sampling chamber and enhances the retention of particles having dimension smaller than those retained by the mesh screen.

In various instances, the housing includes an outlet area, comprising the downstream wall, set downstream of the sampling chamber. The outlet area is advantageously a raised opening at the back of the sampling chamber allowing a liquid to flow through, to leave the housing in the main direction. In various instances, the housing is liquid tight.

The housing can also advantageously include a cover, which is in various instances removable from the housing, comprising a strip fixed to the cover and protruding downwards to the cover, the strip being parallel to the upstream wall. The housing can comprise a grid fixed to the cover and protruding downwards to the cover, the grid being parallel to the strip. Such an arrangement, with the grid located at the outlet area allows to avoid the entrance of fishes and macroinvertebrates inside the sampling chamber.

According to various other embodiments, the strip can be disposed downstream to the upstream wall, the grid being located at the outlet area above the downstream wall.

The cover has in various instances a shape fitting the combination of the inlet, the sampling chamber and the outlet area comprising the downstream wall set downstream to the sampling chamber, with two bended walls going downwards that cover the external side of the respective side walls of the sampling chamber.

According to such exemplary embodiments, the strip is protruding downwards, the downward end of the strip being located lower than the upper end of the upstream wall.

Such an arrangement, with a shifted position of the strip and the upstream wall, allows the stream flow of a liquid to pass over the upper end of the upstream wall and, owing to the strip, to be guided into the main direction and also allowing the retention of finer particles than those retained by the mesh screen.

According to some exemplary embodiments, each extremity of the upstream wall can be fixed respectively to each of the corresponding side walls of the channel by at least one fixing wall.

The at least one support means can comprise an upper part and a lower part, the lower part being in various instances fixed on a bottom wall of the channel and the at least one support means is extending radially.

It should be emphasized, that the length of the support means is smaller than the height of the respective side wall, to position the cover. In the context of the invention, the at least one support means is essentially perpendicular to the liquid main direction, meaning that very slight angular variations are allowed without impairing the whole structure of the housing.

The at least one supporting means is advantageously provided with a corresponding at least one detecting means elevating means, that allow the at least one detecting means to be at a predetermined distance, for example of from 3 mm to 10 mm, from the bottom wall of the channel.

Such at least one elevating means can be fixed at any portion of the at least one supporting means, but is in various instances fixed at the area defined by the lower part of the at least one support means, more in various instances at the bottom end of the lower part, in contact with the bottom wall.

The housing is provided on each side walls with at least one fixation means which is an anchorage point. Each anchorage point can be fixed below the bottom of the side walls. With these anchorage points, the housing can be secured to the sampling environment bed by inserting any kind of spike with a curved end. The number of fixations means on each side wall is not limited, and can be of from 1 to 4.

According to various embodiments, the channel can include at least one cover fixation means, fixed onto the bottom wall of the sampling chamber, adapted to cooperate with at least one corresponding hole of the cover.

The invention also relates to a device including the housing of the invention and at least one detecting means, the at least one detecting means, comprising a frame including two membrane supporting means, wherein a membrane is clamped therebetween.

The frame can comprise at least two fixation means adapted to make the two membrane supporting means clamped each to the other for supporting the membrane.

According to an exemplary advantageous embodiment of the device, the at least one detecting means can be fixed at a predetermined distance from the bottom wall by at least one supporting means, cooperating with at least one corresponding hole arranged in the frame, and at least one corresponding elevating means, and the at least one detecting means is essentially parallel to the bottom wall. Some very slight angular variations are allowed without impairing the whole structure and function of the housing.

Advantageously, the device includes at least two detecting means separated one from the other by a predetermined distance, for example of from 1 cm to 10 cm, in the main direction.

According to an exemplary advantageous embodiment of the device, the at least one detecting means is a passive sampler including a membrane with a sorbing material adapted for retention of chemicals thereon.

The structure of the sorbing material is not limited, and can depend on chemicals to be detected in the flow of the liquid. Typically, the material is a support of polymeric reversed-phase adsorbent, for example in C18-silica, or activated carbon support.

The chemicals that can be retained by the sorbing material are typically selected from the group consisting of pesticides, volatile organic compounds, aromatic derivatives, pharmaceuticals, alkanes, ketones, and aldehydes.

The device can advantageously be used to determine and quantify organic pollutants in some liquid streams that can be rivers and other water streams.

### DRAWINGS

FIG. 1 is a perspective view of the open version (without the hard cover) of the housing according to various embodiments of the invention.

FIG. 2 is a perspective view of the up-side-down hard cover according to various embodiments of the invention.

FIG. 3 is a lateral view of the closed housing according to various embodiments of the invention.

FIG. 4 is a top view of the device including detecting means according to various embodiments of the invention.

FIG. 5 is a top view of the housing with various screen mesh according to some exemplary embodiments of the invention, with various number of detecting means and with various configurations of supporting means.

FIG. 6 is a rear view of the housing according to various embodiments of the invention.

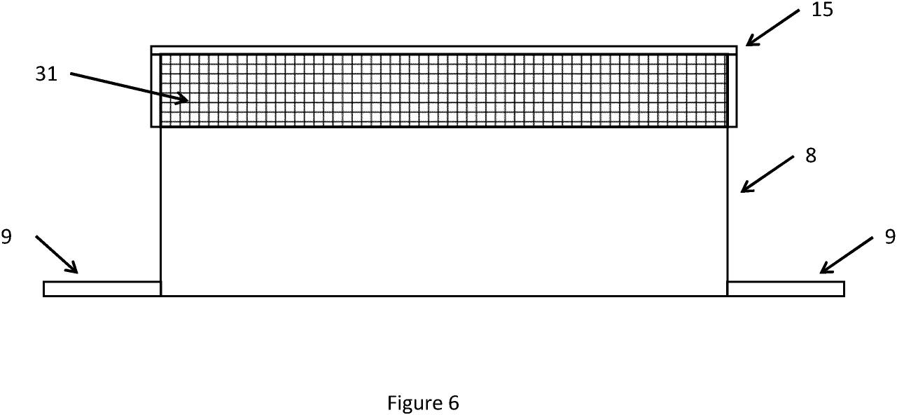

FIG. 7 is a histogram of the recoveries of several organic contaminants obtained with the presented embodiment and with a commercially available deployment housing.

FIG. 8 is a histogram of the relative standard deviation of several organic contaminants obtained with the presented embodiment and with a commercially available deployment housing.

### DETAILED DESCRIPTION

The invention provides a solution when dealing with detecting means or passive samplers exposed in shallow sampling environment and/or having a high turbidity. The invention can also be used in a more classical way, for example in a liquid with a low turbidity and/or during a high flow period.

For example, the housing, and especially the device, can easily be handled for example in a river or in any water environment. The liquid is feeding the sampling chamber where the passive samplers are hosted and where they can stay immersed during low flow or dry periods. The inlet combines a mesh screen and a finer particle retention system including an upstream wall to protect the passive samplers from clogging by smaller foreign materials. The housing includes a removable cover to protect the passive samplers when present from any damages and fixation means to fix the housing or the device either in the sampling environment bed or on a support provided with a quick release system to ease the removal and the installation of the support or the device. The outlet area includes a downstream wall with a raised opening in the sampling chamber to retain a volume of liquid enough to keep the passive samplers, when present, immersed during dry or low flow periods and to let the liquid flow outside during higher flow. In practice, passive samplers are mounted in the sampling chamber parallel to its bottom and secured in position. The hard cover is then closed and secured. The device is placed in the sampling environment with the inlet pointing towards the upstream of the liquid flow and secured by its fixation means or by its support.

When working during a dry or low flow period, the sampling of the liquid will only start when its level will reach the top end of the upstream wall. The liquid will then fill the sampling chamber and the passive samplers will start the collection of the contaminants/pollutants. The liquid leaves the sampling chamber when it reaches the top of the downstream wall of the outlet area. When the level of the liquid in the sampling environment is decreasing and gets below the top of the upstream wall, the sampling chamber is no longer fed and the liquid inside is trapped.

Information about the collections of contaminants by the passive samplers is completed by a series of probes installed inside the chamber allowing the record of the water level in the chamber. The probes are controlled by a PCB located in a hermetic box on top of the housing or of the device and data are stored on a memory flash. PCB and probes are powered by batteries.

When working during the wet season or high flow periods, the presented embodiment is always fully immersed and acts directly like the commercially available deployment housings.

When the deployment time is over, either the passive samplers can be replaced by new ones or the whole housing can be removed from the site.

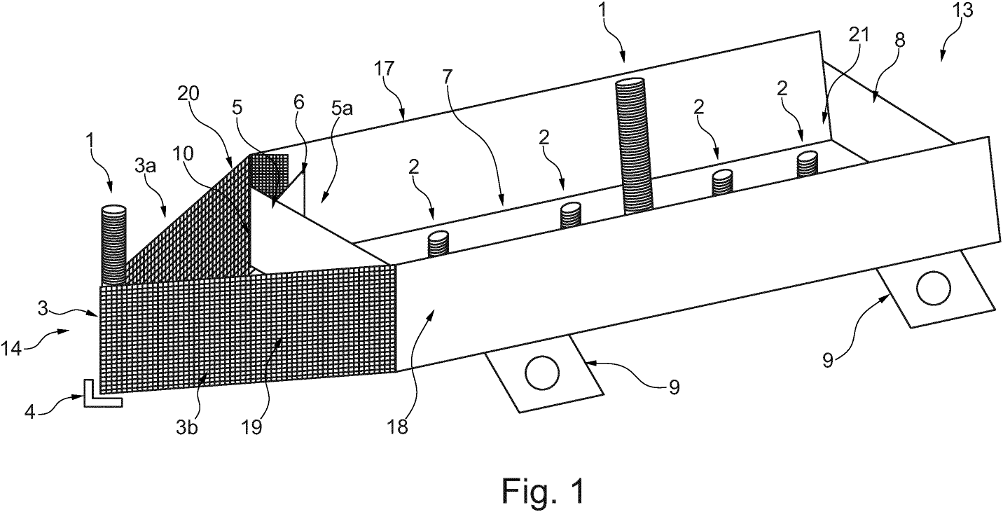

The housing exemplarily depicted in FIG. 1 is made of a material based on stainless steel or based on any other nature, such as glass, polymer, and ceramics. The housing includes a channel for guiding a stream flow of a liquid in a main direction an inlet 14 towards an outlet area 13. A removable mesh screen 3 and a finer particle retention system is arranged at the inlet 14, as well as an upstream wall 5. The outlet area 13 comprises an outlet wall 8 and the housing comprises a sampling chamber 7 delimited by the upstream wall 5 and the outlet wall 8, as well as by two side walls 17, 18. The sampling chamber 7 is very in various instances liquid tight to avoid any liquid loss.

The housing further comprises support means 2 arranged in the sampling chamber 7 and adapted for supporting detecting means (not shown).

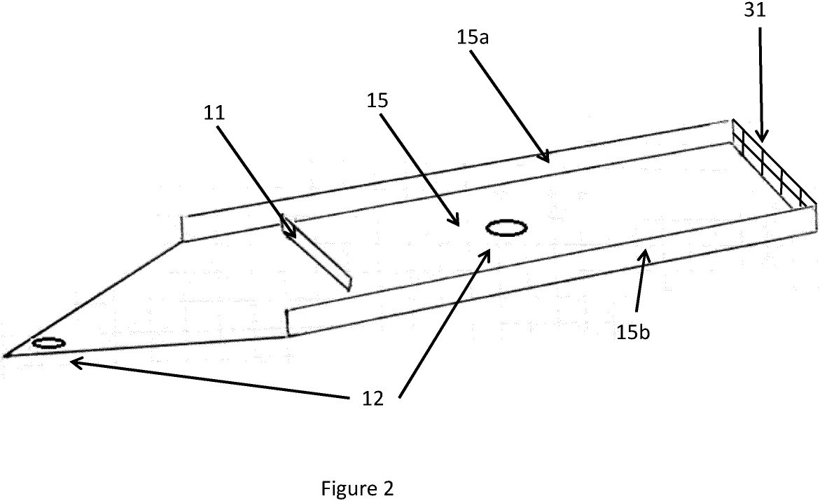

Cover fixation means 1 allow the additional mounting of an optional cover (see 15 on FIG. 2) and extend upwards from a bottom wall 21. Cover fixation means 1 can comprise one means located at the upstream of the inlet means 14 and another means at the middle of the sampling chamber 7. Some additional cover fixation means 1 can be provided, for example one or two more.

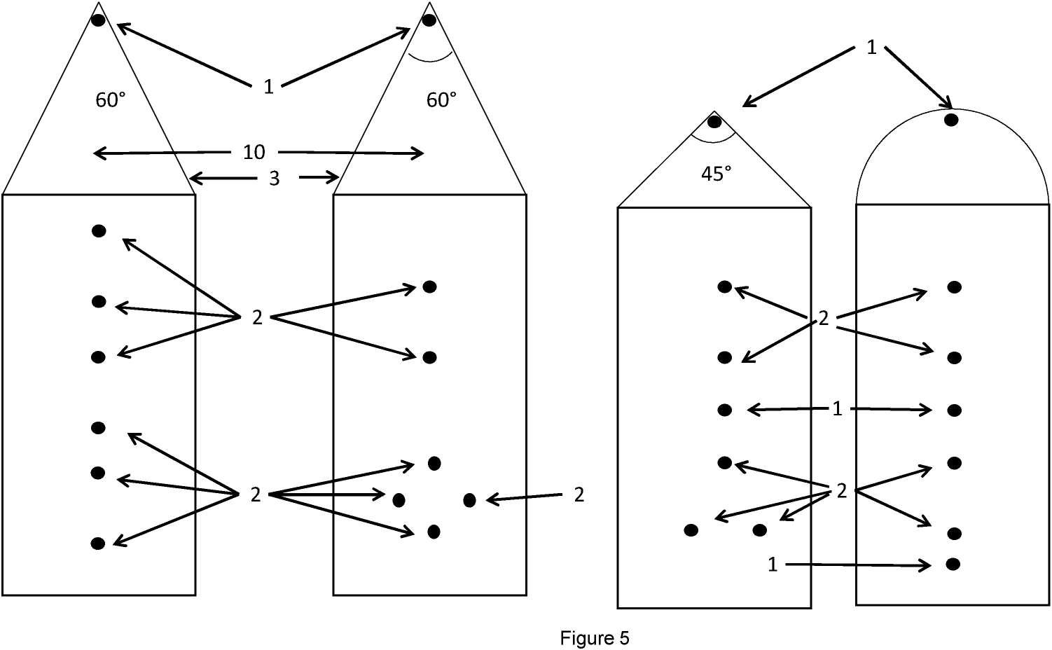

The removable mesh screen 3 comprises an upper end 3a and a lower end 3b and has a V-shaped form presenting two angular walls 19, 20, the angle between the two angular walls 19, 20 being of between about 30° and 70°, in various instances 45° to 60°. Alternatively, the screen mesh 3 can have a ∩ shape (FIG. 5). The screen mesh 3 prevents larger foreign materials from entering in the sampling chamber 7. These shapes allow the larger foreign materials to slide along the removable mesh screen 3 avoiding the clogging of the inlet 14 thereof. Its removable ability is firstly made to exchange the mesh screen 3 and adapt the mesh size according to sampling environment and secondly for maintenance reasons described later. The mesh size is about 5 × 5 mm, in various instances 2 × 2 mm, and the mesh can be of square of circular shape or any other shape that allows the retention of large undesirable materials. The mesh can be disposed perpendicularly to the angular walls 19, 20 or perpendicularly to the plane defined by the upstream wall 5.

As shown in FIG. 1, each cover fixation means 1 is a threaded rod pointing upwards, length thereof being higher than the side walls 17, 18 heights, adapted to guide and secure the hard cover 15 with wing nuts (not shown). The cover fixation means 1 are here located at the middle of the bottom wall 21 between two sets of two support means 2 and at the front part behind the mesh screen 3.

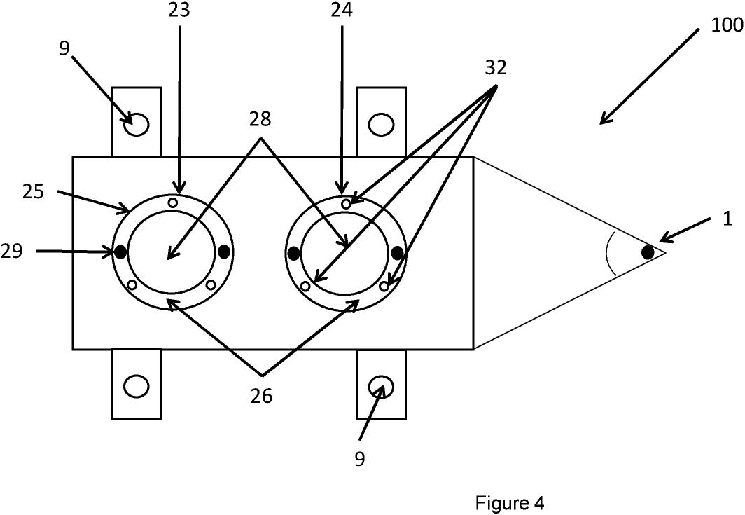

The housing is provided on each side wall 17, 18 with two fixation means 9 which are here two anchorage points (FIG. 4). Each anchorage point 9 consists of a perforated stainless-steel plate fixed below the bottom of the side walls 17, 18. With these four anchorage points 9, the housing can be secured to the sampling environment bed by inserting any kind of spike with a curved end.

A quick release system can ease the removal and the installation of the housing. Alternatively, the anchoring of the housing can be made through a separated part and can consist of a perforated stainless-steel plate with four slides on top (not shown) that allow the hosting of the anchorage points 9 of the housing. The slides are built in a way that the housing cannot slip out backward. The quick release support can have a stainless toggle latch that secures the housing in position. Slides and toggle latch are place in a way that the presented embodiment fits properly in (not shown).

A finer particles sediment trap 10 is confined by the angular walls 19, 20, the bottom wall 21 and the upstream wall 5.

The upper end 3a of the mesh screen 3 presents a height that is greater than the height of the upstream wall 5 depicted by the upper end 5a (FIG. 1). Such a disposition enhances the retention of water in the sampling chamber 7 and enhances the retention of particles having dimension smaller than those retained by the mesh screen 3.

The combination of the mesh screen 3 and the finer particle retention system 10 is acting as a flow buffering system.

The housing includes the sampling chamber 7 which is here a seal tub, delimited by the two side walls 17, 18 and a bottom wall 21 extending from the inlet 14 to the outlet area 13.

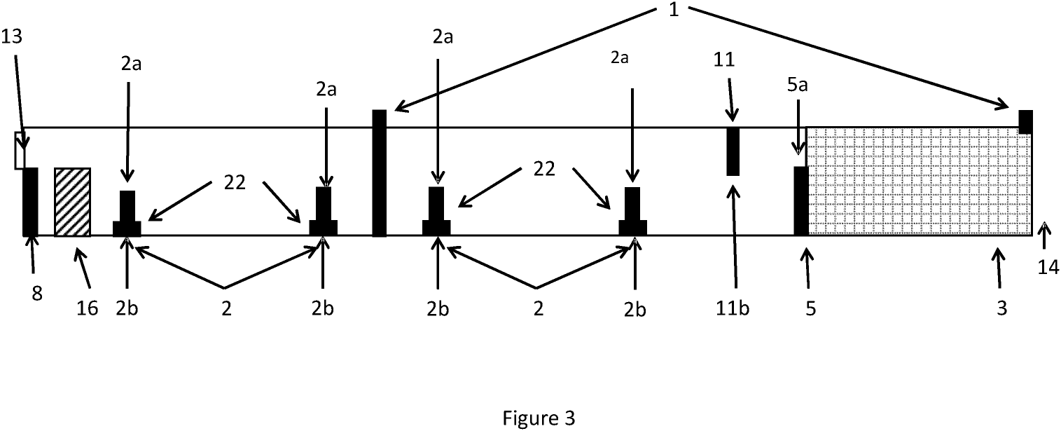

The sampling chamber 7 comprises four support means 2, here being threaded rods, each comprising an upper part 2a and a lower part 2b, the lower part 2b being fixed on a bottom wall 21 of the channel, and each support means 2 is extending radially (FIG. 3). Depending on the configuration of the housing, two supporting means 2 define a set for holding the detecting means 23, 24. The distance between each support means 2 in the same set is of from 5 cm to 15 cm.

The shape of the cover 15 is designed to fit the shape of the assembly of the mesh 3 and the walls 5, 8, 17, 18 and has a spearhead shape (FIG. 2). The cover 15 has along the longer sides, two bended walls 15a, 15b going downwards in order to avoid lateral movement of the cover 15 when installed. The two through holes 12 are designed to host the two threaded rods 1. The two bended walls 15a, 15b help to close and seal the housing. The cover 15 is secured by two wing nuts (not shown) screwed at each threaded rod 1. On the inner side of the cover 15, is the strip 11 of the finer particle retention system. The strip 11 is located backwards than the upstream wall 5 in order to let the liquid from of the sampling environment flows between them. The downward end 11b of the strip 11 is located lower than the upper end 5a of the upstream wall 5 (FIG. 3). The grid 31 is located at the outlet area above the downstream wall 8.

The function of the strip 11 is to block the floating materials that would have passed above the upper end 5a of the upstream wall 5. Another function of the strip 11 is to reroute the liquid from the sampling environment towards the bottom of the sampling chamber 7 to ensure a good turnover of the liquid in the sampling chamber 7.

The strip 11 cooperates with the upstream wall 5 to block the finer sediments that passed through the mesh screen 3. The sediments are then collected in the sediment trap 10.

The length of each support means 2 is shorter than the width of the side wall 17, 18 of the housing, to allow the cover 15 to be fixed on the upper part of the housing (FIG. 3). As seen in FIG. 1, each support means 2 is a threaded rod pointing upwards to host and secure the detecting means 23, 24. For example, the length of each supporting means 2 can vary form 1.5 cm to 4 cm. In FIGS. 1 & 3, the sampling chamber 7 can host up to two detecting means 23, 24 but this configuration is not restrictive as it can be adapted to host more, for example three detecting means 23, 24 (FIG. 5). The two sets of support means 2 are separated each from the other by a predetermined distance which corresponds to the shape of the detecting means 23, 24 and of the housing dimension. Typically, the distance can vary from 5 cm to 30 cm.

Each support means 2 is provided with a corresponding detecting means elevating means 22, that allow the detecting means 23, 24 to be at a predetermined distance from the bottom wall 21 of the channel (FIG. 3).

Each elevating means 22 is fixed at the bottom end of the lower part 2b, in contact with the bottom wall 21.

As previously mentioned, the role of the sampling chamber 7 is firstly to host the detecting means 23, 24 and secondly to collect and keep enough volume of liquid from the sampling environment to fully immerse the detecting means 23, 24 in order to enable the monitoring of the pollutants and keep the detecting means 23, 24 immersed during the dry periods.

The width of the upstream wall 5 can be smaller than the width of the sampling chamber 7 by twice the thickness of the mesh screen 3. With such an upstream wall 5 width, the mesh screen 3 can be inserted and secured by the upstream wall 5, the side walls 17, 18 of the sampling chamber 7 and the positioning means 4 having a L shape placed at the front and bottom of the mesh screen 3. To ensure the sealing of the sampling chamber 7, each of the lateral sides of the upstream wall 5 are respectively fixed to each of the corresponding side walls 17, 18 of the channel by two corresponding fixing walls 6.

The outlet area 13 of the housing is made of the downstream wall 8 of the sampling chamber 7 and the cover 15. The downstream wall 8 is purposively made shorter than the upper edges of both sidewalls 17, 18 of the sampling chamber 7 in order to let an opening when the cover 15 is installed to allow the liquid from the sampling environment to leave the sampling chamber 7 (FIGS. 1, 3 & 6). The grid 31 will block the access to the sampling chamber 7 to any fishes or macroinvertebrates. The grid 31 located at the outlet area has a mesh size in various instances higher or equal to 5 x 5 mm.

Data feeding the additional information collection system are recorded from probes 16 (FIG. 3) located in the sampling chamber 7 near by the downstream area 13 and the downstream wall 8. These probes 16 are measuring the water level inside the sampling chamber 7 at a frequency defined in the code uploaded in a microcontroller (not shown) and the corresponding data are recorded on a support media, both located outside the sampling chamber 7 in a hermetic box (not shown). Probes 16, microcontroller and data storage are powered by an external battery also located in the hermetic box (not shown).

The water level probe will monitor the height of the water in the sampling chamber 7 and record the time where the passive samplers 23, 24 were fully immersed in flowing water, the time they were fully immersed in still water and the time they were fully emerged out of the water.

This additional information collection system can collect any other relevant data with adequate probes.

The FIG. 4 is a top view of the device 100 (without the cover 15) including the housing and two passive samplers 23, 24, comprising a frame 25 with two membrane supporting means 26, 27 (27 not visible), having a circular shape, diameter of which can vary of from 7 cm to 20 cm, two membranes 28 being clamped therebetween. The membranes 28 have the same shape as the frame 25 and sequester the sorbing material. The sorbing material can be a support of polymeric reversed-phase adsorbent, for example in C18-silica, or activated carbon support. The chemicals that can be retained by the sorbing material are typically selected from the group consisting of pesticides, volatile organic compounds, aromatic derivatives, pharmaceuticals, alkanes, ketones, and aldehydes.

The frame 25 comprises three fixation means 32 adapted to make the two membrane supporting means 26, 27 clamped each to the other for supporting the membrane 28.

The two distinct passive samplers 23, 24 are fixed at a predetermined distance from the bottom wall 21 by two threaded rods 2 and two respective elevating means 22, and are cooperating with two corresponding through-hole 29 arranged in the frame 25. The fixation of the passive samplers 23, 24 on the supporting is realised by nuts (not shown). The two passive samplers 23, 24 are parallel to the bottom wall 21.

The purpose of the device 100 is to be used in shallow sampling environments having a high turbidity, during dry or low flow periods punctuated by flush events rising the liquid level of the sampling environment. This purpose is not restrictive as the device can be used fully submerged during the whole deployment time in a sampling environment having a low turbidity.

For the first deployment period, the installation of the device consists, in the present configuration, of:
installing at least one passive sampler 23, 24 in the housing at their dedicated places by fixing them to the threaded rods 2, comprising the elevating means 22, with nuts,
closing the device by inserting the two threaded rods 1 through the cover 15 and securing it by screwing a wing nut to each rod 1,
installing the device in the sampling environment with inlet 14 pointing upstream and securing it with its anchorage points 9.

For the following deployments at the same location, the device can be let in place and the passive samplers 23, 24 exchange by simply removing the hard cover 15.

Hereafter is described the operating principle of the housing or of the device 100 for the dry or low flow sampling environments punctuated by flush events and for the high flow sampling environments.

The primarily purpose of housing or of the device is to be used in shallow sampling environments having a high turbidity i.e., during dry or low flow periods punctuated by flush events.

During dry or low flow periods, the liquid level of the sampling environment is respectively inexistent or too low to reach the top of the upstream wall 5. Therefore, the sampling chamber 7 is disconnected from the sampling environment and the monitoring of the pollutant does not occur.

When a flush event occurs, the liquid level of the sampling environment is rising, carrying different kinds of foreign materials and suspended sediments. The coarse foreign materials are blocked by the mesh screen 3 and slide along because of its specific shape while the smaller materials are blocked by the finer particle retention system, the heavier materials by the upstream wall 5 and the floating materials by the strip 11 of the cover 15. When the liquid level of the sampling environment reaches the top of the upstream wall 5a, the liquid from the sampling environment, cleaned of a good part of its foreign materials, starts to fill the sampling chamber 7. When the passive samplers 23, 24 get in contact with the liquid, the accumulation of the contaminants onto the sorbing material of the membrane 28 starts. The liquid from the sampling environment leaves the sampling chamber 7 when it reaches the top of the outlet area 13 in the downstream area 8. The flow of the liquid from the sampling environment through the device allows the renewing of the cleaned liquid from the sampling environment in the sampling chamber 7 and therefore the pursuit of the accumulation of the contaminants in the passive samplers 23, 24. The treatment of the liquid from the sampling environment avoids the accumulation of foreign materials on the top of the membrane 28 and thus, the decrease of the contaminant uptake rate.

At the end of the flush event, the liquid level is decreasing and when it gets bellow the top of the upstream wall 5, the sampling chamber 7 is no longer fed but the passive samplers 23, 24 stay immersed for a period depending of the weather conditions. Therefore, the passive sampling mode changes from the turbulent one to the quiescent one. The uptake rates of the contaminants will decrease with their depletion in the sampling chamber 7 until it becomes negligible. Even if these conditions are not representative of the external conditions, this effect is limited since the depletion of organic pollutants will be completed within a few hours due to a limited volume of sampling chamber 7.

During the next flush event, the new incoming liquid will replace the old one and the passive sampling starts again. As the membrane 28 from the passive samplers 23, 24 stay hydrated, an increase of the contaminant uptake rate due to the hydration of the membrane 28 will be avoided, as the accumulation process will stay controlled by diffusion.

The water level probe 16 provides the information on how long lasted the turbulent adsorption mode, the quiescent adsorption mode and the periods where the passive samplers 23, 24 were out of the water. These data will allow a better understanding of the adsorbed masses on the passive samplers 23, 24.

### EXAMPLE

A comparative test was performed where a commercially available device (EST-Lab) was equipped with two passive samplers containing OASIS HLB (Waters) as adsorbing material and where the device according to the invention was equipped with two passive samplers of the same kind. Both devices were exposed at the same time and at the same sampling point. The selected organic contaminants cover a wide polarity range with log Kow values from 0.66 to 3.74. The recovery of each organic contaminant collected via the device of the invention was calculated based on the one from the commercially available device.

This comparison has been made under field conditions in a mid-mountainous river under steady flow conditions with an exposure time of 14 days.

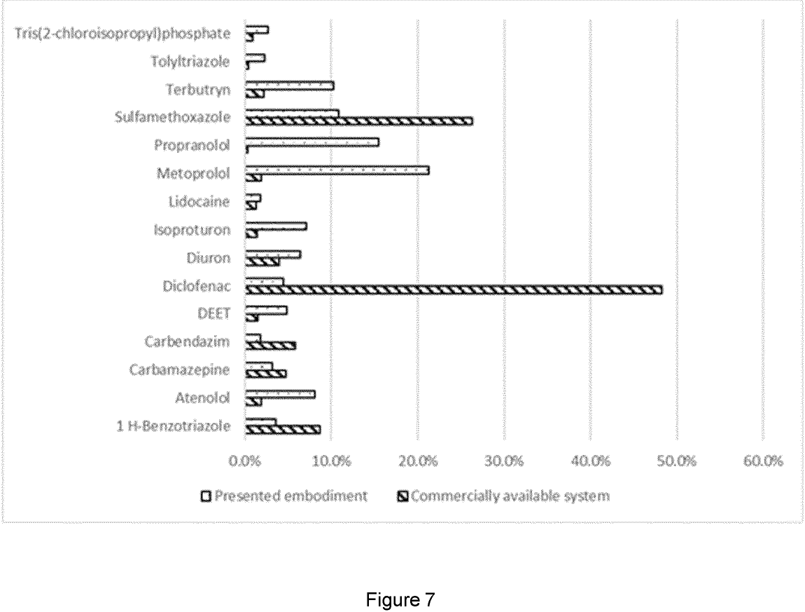

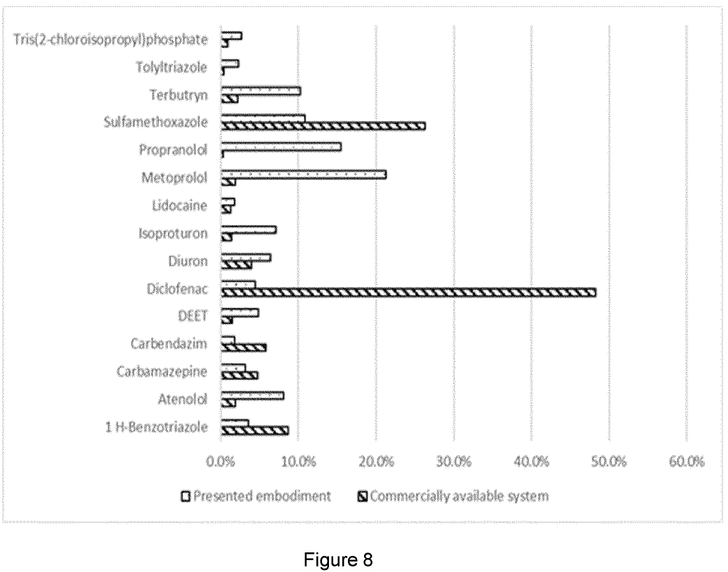

The results are presented in FIG. 7, the relative standard deviation in FIG. 8. The recoveries from the device of the invention range from 68% to 104% with an average recovery value of 85%. Even if the values are a bite lower than for the commercially available deployment housing, they stay acceptable.

For each deployment housing, the relative standard deviations were less than 10% with a couple of exceptions (see FIG. 8).

1-17. (canceled)

18. A passive sampler deployment housing, said housing comprising:
a channel for guiding a stream flow of a liquid in a main direction, the channel having two side walls and one downstream wall, defining a sampling chamber; and
at least one support means adapted for supporting at least one detecting means and arranged in the channel, the downstream wall, disposed essentially perpendicular to the liquid main direction, having a height such that the flow of the liquid is accumulated in the channel at a level that is sufficient to overlap the at least one support means and optionally the at least one detecting means.

19. The housing according to claim 18, including an inlet, disposed upstream of the channel, the housing comprising a mesh screen delimiting an upper end and a lower end having a V-shaped form, presenting two angular walls, the angle between the two walls is of from 45° to 80°, having a ∩ shape or an arc of circle shape.

20. The housing according to claim 19, wherein the inlet includes an upstream wall disposed at an upstream position with regard to the at least one support means, the upstream wall is perpendicular to both side walls of the channel.

21. The housing according to claim 18, including a cover removable from the housing, comprising a strip fixed to the cover and protruding downwards to the cover, the strip being parallel to the upstream wall.

22. The housing according to claim 21, wherein the strip is disposed downstream to the upstream wall.

23. The housing according to claim 21, wherein the cover has a shape fitting the combination of the inlet, the sampling chamber and an outlet area comprising the downstream wall set downstream to the sampling chamber, with two bended walls going downwards that cover the external side of the respective side walls of the sampling chamber.

24. The housing according to claim 21, wherein the downward end of the strip is located lower than the upper end of the upstream wall.

25. The housing according to claim 21, comprising a grid fixed to the cover and protruding downwards to the cover, the grid being parallel to the strip.

26. The housing according to claim 21, wherein the at least one support means comprises an upper part and a lower part, the lower part being fixed on a bottom wall of the channel, and the at least one support means is extending radially.

27. The housing according to claim 21, wherein the at least one supporting means is provided with a corresponding at least one detecting means elevating means, that allow the at least one detecting means to be at a predetermined distance from the bottom wall of the channel.

28. The housing according to claim 27, wherein the at least one detecting means elevating means is fixed at any portion of the length of the at least one supporting means in contact with the bottom wall (21).

29. The housing according to claim 28, wherein the at least one detecting means elevating means is fixed at the area defined by the lower part of the at least one support means.

30. The housing according to claim 21, the housing being provided on each side walls with at least one fixation means fixed below the bottom of the side walls.

31. The housing according to claim 21, wherein the channel includes at least one cover fixation means, fixed onto the bottom wall of the sampling chambe, adapted to cooperate with at least one corresponding hole of the cover.

32. A device including the housing of claim 21 and at least one detecting means, the at least one detecting means comprising a frame including two membrane supporting means, and a membrane, comprising a sorbing material, which is clamped therebetween.

33. The device according to claim 32, wherein the frame comprises a least two fixation means adapted to make the two membrane supporting means clamped each to the other for supporting the membrane.

34. The device according to claim 32, wherein the at least one detecting means is fixed at a predetermined distance from the bottom wall by at least one supporting means, cooperating with at least one corresponding hole arranged in the frame, and at least one corresponding elevating means, and the at least one detecting means is essentially parallel to the bottom wall.

35. The device according to claim 32, comprising at least two detecting means separated one from the other by a predetermined distance (d) in the main direction.
